(19) United States
(12) Patent Application Publication
(10) Pub. No.: US 2007/0014702 A1
(43) Pub. Date: Jan. 18, 2007

(54) Track light fan

(76) Inventors: Michael J. Cruz, Las Vegas, NV (US); Tracie L. Cruz, Las Vegas, NV (US)

Correspondence Address: Galasso & Associates, LP, P.O. Box 26503, Austin, TX, 78755-0503, US

(21) Appl. No.: 11/481,463

(22) Filed: Jul. 6, 2006

Related U.S. Application Data
(60) Provisional application No. 60/699,537, filed on Jul. 15, 2005.

(51) Int. Cl. A61L 9/12

(52) U.S. Cl. 422/124; 416/62; 416/146 R

(57) ABSTRACT

The Track Light Fan consists of a fan motor covered by a housing, a fragrance slot and fragrance center, a speed adjustor, a plurality of rotating blades covered by a protective grate, a track light connector, and a rod for connecting the fan to the track light connector. To use The Track Light Fan, a user simply attaches the track light connector to a standard track. The operator can then use the speed adjustor and other knobs and buttons to control the device.

### CROSS REFERENCE TO RELATED APPLICATIONS

This Non-Provisional Patent Application claims priority to U.S. Provisional Patent Application Ser. No. 60/699,537 entitled “The Track Light Fan” filed on Jul. 15, 2005.

### FIELD OF THE DISCLOSURE

The disclosures made herein relate generally to the ceiling fan industry. The invention discussed herein is in the general classification of ceiling light fans.

### BACKGROUND

Many individuals utilize track lighting in their homes, businesses or other establishments. Conventional ceiling lights are often attached to fans, however, track lighting systems rarely, if ever, have a fan attachment option. The conventional ceiling light fans are also large and occupy a substantial amount of space. Installing these types of fans can also be cumbersome.

Hence, there is a need in the art for an easy to use, inexpensive and effective device that will attach to a track light and allow circulation of air throughout a room or other area.

### SUMMARY OF THE DISCLOSURE

The Track Light Fan consists of a fan motor covered by a housing, a fragrance slot and fragrance center, a speed adjustor, a plurality of rotating blades covered by a protective grate, a track light connector, and a rod for connecting the fan to the track light connector. To use The Track Light Fan, a user simply attaches the track light connector to the standard track. The operator can then use the speed adjustor to choose the appropriate fan blade speed.

The principal object of this invention is to provide a device that attaches to a track lighting system and circulates air.

Another object of this invention is to provide a device that circulates air and freshens the smell of a room.

Another object of this invention is to provide an affordable device for circulating air and freshening a room.

Another object of this invention is to provide an easy to use, adjustable device for circulating fresh air throughout a room or other area.

Yet another object of this invention is to provide an easy to install device for circulating and freshening air in a room or other area.

### BRIEF DESCRIPTION OF THE DRAWINGS

FIG. 1 depicts a front view of the preferred embodiment of the invention.

FIG. 2 depicts a perspective view of the preferred embodiment of the invention.

### DETAILED DESCRIPTION OF THE DRAWINGS

The preferred embodiment of The Track Light Fan is comprised of at least some of the following: a track with lights, a fan motor covered by a housing, rotating blades covered by a protective grate, a fragrance slot and fragrance center, a speed adjustor, an oscillation adjustor, an override switch, a track light connector, and a rod for connecting the fan to the track light connector.

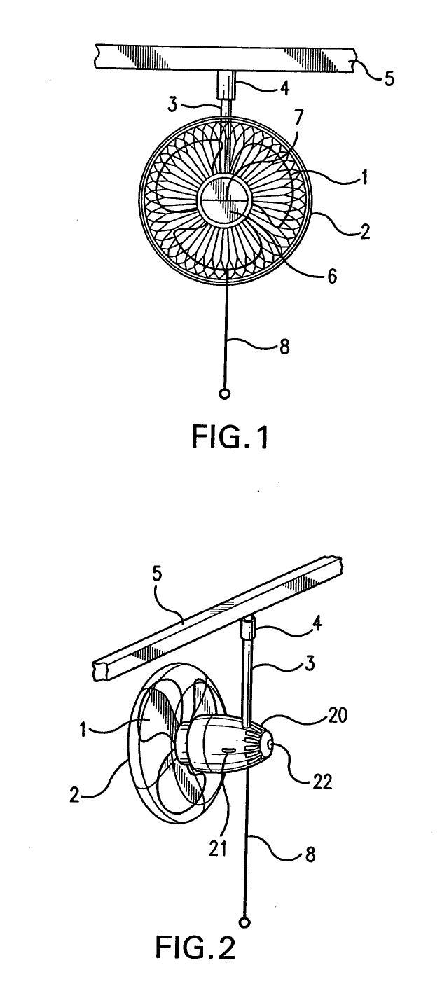

In the preferred embodiment of the invention, shown in FIG. 1, a housing (not pictured) covers a motor (not pictured). The motor is operatively connected to the fan blades 1 located within a grate 2. The fan blades 1 and grate 2 are made of metal in this preferred embodiment though they could easily be made of plastic or other suitable materials. The housing connects via a rod 3 to a track connector 4. The track connector 4 fits into a socket in the track 5. The grate 2 is circular and approximately eight inches in diameter and the housing and grate 2 extend approximately four and a half inches in depth. The rod 3 connects through the rear and top of the housing and is made of metal and is approximately six inches in length. The rod 3 contains the appropriate wiring to supply power to the motor.

In the center of the grate 2, a fragrance center 6 that is circular in shape has a fragrance slot 7 extending through the center diameter that allows a selected fragrance to emanate throughout the room. The fragrance center 6 is made of plastic and is approximately three inches in diameter. A variety of fragrances are available for use with the preferred embodiment of this device. For example, apple, vanilla, coffee, cherry, and piña colada are just a few of the fragrances available. A speed adjustor chain 8 hangs from the rear and bottom of the housing. It is approximately two feet in length and made of metal.

FIG. 2 shows a perspective view of the preferred embodiment of the invention. The housing 20 that encloses the motor can be seen. The housing is made of plastic and has an oscillation adjustor knob 21 on its exterior that can stop or start the oscillation of the fan. An override switch 22 is located on the rear of the housing 20. This view also shows the grate 2, fan blades 1, rod 3, track connector 4, track 5 and speed adjustor chain 8 in greater detail.

To use The Track Light Fan, a user simply chooses a location to install the device and a fragrance appropriate for the location. After connecting the track connector to the appropriate track and inserting a suitable fragrance center, the user can adjust the speed and oscillation of the device with the speed adjustor chain and oscillation adjustor knob. The fan blades will rotate and fragranced air will circulate throughout the room. If a different fragrance is desired or it is time for the fragrance center to be replaced, the fragrance center can be removed from the center of the grate and a suitable replacement fragrance center can be inserted.

The components of The Track Light Fan may vary widely but will likely use metal, plastic and electrical components. The metals would ideally be selected from available steel or alloys of steel and aluminum. The production process related to the use of these metals insures that the metal is non-corrosive, durable and strong. The selected metal should have high impact strength and be capable of accepting and retaining coloring materials for an extended length of time.

The plastic used in the production will ideally be selected for durability and longevity. Thermoplastics are commonly used in the manufacturing of components similar to those used in this invention. Polyethylene, polypropylene, and other similar thermoplastic materials would be among those with the necessary traits. Members of this family are recognized universally as being versatile and of high quality.

The plastic components of The Track Light Fan can also be formed with the use of plastic molding techniques, such as injection molding or blow molding. Injection molding requires melted plastic to be forcefully injected into relatively cool molds. As the plastic begins to harden, it takes on the shape of the mold cavity. This technique is ideal for the mass production of products. Alternatively, blow molding, a form of extrusion, could be utilized. Blow molding involves a molten tube being pushed into a mold. Compressed air then forces the molten tube against the cold walls of the mold.

All electronic components of the invention will also be ideally selected from those currently having the highest industry ratings. These components will also meet and/or exceed all safety and usage regulations. Wiring and associated connecting hardware should be insulated and otherwise protected from intrusion by any harmful or degrading elements, including water, medium level temperatures, and low to medium impact force.

It should be obvious that the components of the present invention can be of various shapes and sizes. It should also be obvious that the components of the invention can be made of different types of metals, plastics or other suitable materials and can be of any color. It should further be obvious that although the present invention is ordinarily used within a room, it could be used in any suitable area.

It will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A fan comprising:
(a) a housing containing a motor operatively connected to a set of blades;
(b) a grate covering the set of blades; and
(c) a rod connecting to the housing having a track connector to be inserted into a socket of a track lighting device;

2. The fan of claim 1 wherein the housing is plastic.

3. The fan of claim 1 wherein the housing is metal.

4. The fan of claim 1 wherein the set of blades are plastic.

5. The fan of claim 1 wherein the set of blades are metal.

6. The fan of claim 1 wherein the grate is metal.

7. The fan of claim 1 wherein the grate is plastic.

8. The fan of claim 1 wherein the rod is metal.

9. The fan of claim 1 further comprising
a fragrance center containing a fragrance located on the grate.

10. The fan of claim 9 wherein the fragrance center is circular in shape and has a fragrance slot extending through the center diameter.

11. The fan of claim 9 wherein the fragrance center is made of plastic.

12. The fan of claim 9 wherein the fragrance is apple.

13. The fan of claim 9 wherein the fragrance is vanilla.

14. The fan of claim 9 wherein the fragrance is coffee.

15. The fan of claim 9 wherein the fragrance is cherry.

16. The fan of claim 1 further comprising
a speed adjustor mechanism connecting through the bottom of the housing.

17. The fan of claim 16 wherein the speed adjustor mechanism is a chain.

18. The fan of claim 1 further comprising
an oscillation adjustor mechanism located on the exterior of the housing.

19. The fan of claim 18 wherein the oscillation adjustor mechanism is a knob.

20. The fan of claim 1 wherein a cord with a plug provides power to the motor.

21. The fan of claim 1 further comprising
an override switch located on the housing.

22. A fan comprising:
(a) a housing made of metal containing a motor operatively connected to a set of blades made of metal;
(b) a grate made of metal covering the set of blades;
(c) a rod made of metal operatively connecting through the housing having a track connector to be inserted into a socket of a track lighting device;
(d) a fragrance center that is circular in shape and located on the center of the grate containing a fragrance and having a fragrance slot extending approximately the diameter of the fragrance center;
(e) a speed adjustor chain connecting through the housing and an oscillation adjustor knob located on the housing; and
(f) an override switch located on the housing.
